SEQUENCE LISTING (32 sequences)

SEQ ID NO: 1 - DNA, 4155 bp, Arabidopsis thaliana
  CDS (1)..(4152)
atgagtagtttggtggagaggcttcgcatacgatctgataggaaaccagtttataacctagatgattctgatgatgacgacttcgttcctaaaaaagatcgaacctttgagcaagtcgaggctattgtcagaactgatgcgaaagaaaatgcatgtcaggcttgtggggaaagtactaatcttgtaagctgcaatacatgcacttatgcgttccatgctaaatgcttagttccacctcttaaagatgcttccgtggaaaattggagatgccctgaatgtgttagtcctcttaacgagatagataagatattggattgtgaaatgcgtcctacaaaatctagtgaacaaggttcctccgatgcggaaccgaagccaatttttgtgaaacagtatctcgtgaagtggaagggattatcataccttcactgctcttgggtgcctgagaaggagttccagaaggcttataagtcaaatcatcgtttaaaaaccagagtgaacaattttcaccgtcaaatggagtcattcaataacagcgaagatgattttgttgccatacgtcctgagtggaccactgttgatcggattcttgcctgcagagaggaagatggggagctggaatatcttgtcaaatataaagagctatcctatgatgaatgttattgggagtcagaatcagacatctcaaccttccagaatgaaattcaaaggttcaaggatgtaaattctagaactcgcagaagtaaagatgttgaccataaaagaaatcccagagactttcaacagtttgatcatactcctgaattcctcaaaggcttgttacatccataccagcttgagggacttaattttttgcggttctcgtggtcaaaacagacgcatgtaatccttgctgatgaaatgggactaggcaagacaattcaaagcattgcccttttagcttcactttttgaggagaacctcattccgcatttggtaattgctcctctatcgactctgcgtaactgggagagagagtttgccacatgggccccacagatgaacgtggttatgtattttggcactgcgcaagctcgagcagttatcagagaacatgagttttacttatcgaaagatcaaaaaaagatcaagaaaaagaaatctggacaaataagtagcgaaagcaagcaaaaaagaatcaagtttgatgtcctcctcacatcgtatgagatgatcaacctagattcagcagttctaaaaccaattaagtgggagtgcatgattgttgatgaaggtcatcgactgaaaaataaggattcaaagctgttctcttcattgacacagtattcaagtaaccaccgtattcttctgacaggaacaccacttcagaacaacttggatgaacttttcatgctcatgcattttcttgatgcggggaagtttggaagtttggaggagttccaggaggagttcaaagatattaatcaagaggagcagatctcaaggttgcacaaaatgttggctccacatttgctcagaagggtaaaaaaagacgtaatgaaagacatgccccccaaaaaggagctcattttgcgtgttgatctgagcagtctgcagaaagaatattacaaagctatttttacccgtaattatcaagtattgacaaaaaagggaggtgctcaaatttcccttaataacattatgatggaattacgaaaagtatgctgccatccttatatgctagagggtgttgagccagttattcacgacgcaaatgaagctttcaaacaacttttggagtcttgtggaaagctgcaacttctagataaaatgatggtcaaactgaaagagcaaggacacagagtcctaatatacacacagtttcagcatatgctggacttacttgaagactactgtacccataagaaatggcagtacgagcgaattgatggaaaggttggcggagctgagcggcaaatacgcatagatcggttcaatgccaaaaattctaacaagttttgttttttgctctccacaagagctggtggcttaggaataaatcttgcaacggctgatacagtaatcatttatgacagtgactggaatcctcatgctgatcttcaagcaatggctagagctcatcgacttggccaaacaaataaggtgatgatttataggctcataaaccgaggcaccattgaagaaaggatgatgcaattgactaaaaagaaaatggttctagagcatcttgttgttgggaaactcaaaacacaaaacattaatcaggaagagttagatgacatcatcaggtatggatcaaaggagctttttgctagtgaagatgatgaagcaggaaagtctggaaaaattcattatgatgatgcggctatagacaaattgcttgatcgtgatctcgtggaggcagaggaagtctcagtggatgatgaagaggagaatggattcttaaaggctttcaaggtggctaattttgaatatatagatgaaaatgaggcagcagcattagaggcacagagagtcgctgctgaaagcaaatcttcagcaggcaattctgatagagcaagttattgggaagagttgttaaaagataaatttgagctgcaccaggctgaggagcttaatgctcttggaaaaaggaagagaagtcgcaagcagttggtatccattgaagaagatgatcttgctggtttggaagatgtgagctctgatggagatgaaagttatgaagctgagtcaacagatggtgaagcagcaggacaaggagttcagacgggtcgacggccgtacagaagaaagggtcgcgataatttggaaccaactccgttgatggaaggtgaggggagatctttcagagtactgggtttcaaccagagtcaaagggccatttttgtacagactttgatgaggtatggagctggcaattttgattggaaggagtttgttcctcgcttaaagcagaagacctttgaagaaataaatgaatatggaatactcttcttgaagcacattgctgaagaaatagacgagaattctccaaccttttcagatggtgtgcccaaggaaggacttagaatagaagatgttctagtcagaattgctcttctgatactagttcaggagaaggtgaaatttgtagaagatcatccagggaaacctgttttcccctctcgcattcttgaaagattccccggactgagaagtggaaaaatttggaaggaggaacatgacaagataatgatacgtgctgttttaaagcatgggtacggacggtggcaagctattgttgatgacaaagagttggggatccaagagcttatctgcaaagaattgaatttccctcacataagtttgtctgctgctgaacaagctggtttgcaggggcagaatggtagtgggggctctaatccgggagcacagactaaccagaatcctggaagcgttattactgggaacaataatgcttctgctgatggggctcaagtaaactcgatgttctattatcgggacatgcagagacgacttgttgagtttgtgaaaaagcgagttctgcttttggagaaggcgatgaattatgaatacgcagaggaatattatggacttggtggctcatcatctatccctactgaagaaccagaagctgaaccaaagatcgctgacacagtgggagtgagctttattgaggttgatgatgaaatgcttgatggacttcctaagactgatcctatcacttcagaagaaattatgggggctgctgttgacaacaaccaagcgcgggtcgaaatagctcaacattataaccagatgtgcaaacttcttgatgagaacgctcgggaatcagtccaagcatatgtaaacaaccaaccaccgagtaccaaggtgaatgagagcttccgtgcactcaaatctatcaatggtaacattaacacaatcctttcgattacatctgatcaatccaagtcacatgaagacgacaccaagccagacctaaacaatgttgagatgaaggacacggccgaagaaacaaaaccgttaagaggtggcgtcgtcgatctgaatgtggtggagggagaggagaacattgctgaagctagtggaagtgttgatgtaaaaatggaagaagccaaagaagaagagaagccaaagaacatggtcgttgattga

SEQ ID NO: 2 - protein, 1384 aa, Arabidopsis thaliana
MSSLVERLRIRSDRKPVYNLDDSDDDDFVPKKDRTFEQVEAIVRTDAKENACQACGESTNLVSCNTCTYAFHAKCLVPPLKDASVENWRCPECVSPLNEIDKILDCEMRPTKSSEQGSSDAEPKPIFVKQYLVKWKGLSYLHCSWVPEKEFQKAYKSNHRLKTRVNNFHRQMESFNNSEDDFVAIRPEWTTVDRILACREEDGELEYLVKYKELSYDECYWESESDISTFQNEIQRFKDVNSRTRRSKDVDHKRNPRDFQQFDHTPEFLKGLLHPYQLEGLNFLRFSWSKQTHVILADEMGLGKTIQSIALLASLFEENLIPHLVIAPLSTLRNWEREFATWAPQMNVVMYFGTAQARAVIREHEFYLSKDQKKIKKKKSGQISSESKQKRIKFDVLLTSYEMINLDSAVLKPIKWECMIVDEGHRLKNKDSKLFSSLTQYSSNHRILLTGTPLQNNLDELFMLMHFLDAGKFGSLEEFQEEFKDINQEEQISRLHKMLAPHLLRRVKKDVMKDMPPKKELILRVDLSSLQKEYYKAIFTRNYQVLTKKGGAQISLNNIMMELRKVCCHPYMLEGVEPVIHDANEAFKQLLESCGKLQLLDKMMVKLKEQGHRVLIYTQFQHMLDLLEDYCTHKKWQYERIDGKVGGAERQIRIDRFNAKNSNKFCFLLSTRAGGLGINLATADTVIIYDSDWNPHADLQAMARAHRLGQTNKVMIYRLINRGTIEERMMQLTKKKMVLEHLVVGKLKTQNINQEELDDIIRYGSKELFASEDDEAGKSGKIHYDDAAIDKLLDRDLVEAEEVSVDDEEENGFLKAFKVANFEYIDENEAAALEAQRVAAESKSSAGNSDRASYWEELLKDKFELHQAEELNALGKRKRSRKQLVSIEEDDLAGLEDVSSDGDESYEAESTDGEAAGQGVQTGRRPYRRKGRDNLEPTPLMEGEGRSFRVLGFNQSQRAIFVQTLMRYGAGNFDWKEFVPRLKQKTFEEINEYGILFLKHIAEEIDENSPTFSDGVPKEGLRIEDVLVRIALLILVQEKVKFVEDHPGKPVFPSRILERFPGLRSGKIWKEEHDKIMIRAVLKHGYGRWQAIVDDKELGIQELICKELNFPHISLSAAEQAGLQGQNGSGGSNPGAQTNQNPGSVITGNNNASADGAQVNSMFYYRDMQRRLVEFVKKRVLLLEKAMNYEYAEEYYGLGGSSSIPTEEPEAEPKIADTVGVSFIEVDDEMLDGLPKTDPITSEEIMGAAVDNNQARVEIAQHYNQMCKLLDENARESVQAYVNNQPPSTKVNESFRALKSINGNINTILSITSDQSKSHEDDTKPDLNNVEMKDTAEETKPLRGGVVDLNVVEGEENIAEASGSVDVKMEEAKEEEKPKNMVVD

SEQ ID NO: 3 - DNA, 9353 bp, Arabidopsis thaliana
atgagtagtttggtggagaggcttcgcatacgatctgataggaaaccagtttataacctagatgattctgatgatgacgacttcgttcctaaaaaagatcgaacctttgagcaagtcgaggctattgtcagaactgatgcggtttgtttctcctctcgagcttattgttcagcttttactgttttatgtgttctattttaatccttttttttgtgttgttactctgaatttgtagaaagaaaatgcatgtcaggcttgtggggaaagtactaatcttgtaagctgcaatacatgcacttatgcgttccatgctaaatgcttagttccacctcttaaagatgcttccgtggaaaattggagatgccctgaatgtgtaagattttagttacggtccacaattatgttttgggatgctacaggttccatttttcttacatggaagaattgttgtttacatttgcaggttagtcctcttaacgagatagataagatattggattgtgaaatgcgtcctacaaaatctagtgaacaaggttcctccgatgcggaaccgaagccaatttttgtgaaacagtatctcgtgaagtggaagggattatcataccttcactgctcttggtagttactgcgtgtcttttttgctgtctggacacgctaattatcaatgtttctttctgtgaacactataatatgtgatttatttccttttactaatcatagggtgcctgagaaggagttccagaaggcttataagtcaaatcatcgtttaaaaaccagagtgaacaattttcaccgtcaaatggagtcattcaataacagcgaagatgattttgttgccatacgtcctgagtggaccactgttgatcggattcttgcctgcaggtctagagaatggaattaattcctttatttatctatctgccaactttttttttaatatccttgttttcagcataatccattctctaataaacacgtatctttgatagagtgctgcttaacctaaatttactgttatcacgattttgggtctctgaaacatgataaatgacctgcttaccttttttttcttctttttaagttaccattttcttagttgtttcgtaaatcaggaattgtgacagttgcattggtttcttttatgatatagagaggaagatggggagctggaatatcttgtcaaatataaagagctatcctatgatgaatgttattgggagtcagaatcagacatctcaaccttccagaatgaaattcaaaggttcaaggatgtaaattctagaactcgcagaagtaaagatgttgaccataaaagaaatcccagagactttcaacagtttgatcatactcctgaattcctcaaaggtatttggatcaccttaaatcatatactataaatgtttcttatatttggtacttatagatgttatgatttatttgtttcctgcgattgaaggcttgttacatccataccagcttgagggacttaattttttgcggttctcgtggtcaaaacagacgcatgtaatccttgctgatgaaatgggactaggtaatttttcaattgtcccacttgggtggtcacatagatcttttcatccattgtaaggggcctttgttttctattcctgtaatgttgtgagatttttcctgttacaggcaagacaattcaaagcattgcccttttagcttcactttttgaggagaacctcattccgcatttggtaattgctcctctatcgactctgcgtaactgggagagagagtttgccacatgggccccacagatgaacgtggtatgtatgcagttatacacgcaatgatctgtgccatttgtatgtttttgttgtttgttaatggaatggtcttcgtggtcatttgacgggtaggttatgtattttggcactgcgcaagctcgagcagttatcagagaacatgagttttacttatcgaaagatcaaaaaaagatcaagaaaaagaaatctggacaaataagtagcgaaagcaagcaaaaaagaatcaagtttgatgtcctcctcacatcgtatgagatgatcaacctagattcagcagttctaaaaccaattaagtgggagtgcatggtaactcttattctctaatgagactttactttctcttagtcgtctctctttctctcttacatgttgcctagtaacaattgttttgggcagattgttgatgaaggtcatcgactgaaaaataaggattcaaagctgttctcttcattgacacagtattcaagtaaccaccgtattcttctgacaggaacaccacttcaggttcgtcatttgagtttgatttctgaagtttatactttcaatagttgtatctgagcatagtagctacgatttgcaatgagaattgttatatattatcttgcactaatgtcttacctgattagttgcaatatgttactgatgattatgtggtgcctttacagaacaacttggatgaacttttcatgctcatgcattttcttgatgcggggaaggtatcacaagaatagcaaagataaataagttcgcatacttaacagaattttatgtagctaacatgttatttgattgcacaatacttgcagtttggaagtttggaggagttccaggaggagttcaaagatattaatcaagaggagcagatctcaaggttgcacaaaatgttggctccacatttgctcagaagtattaaccaaaactatttgttcatcttttttaatttatatgtgtttcaaaagtttggttggagggaatctttcatagtaataattttatgatcttaaccatgctgtctcgtattttgattgctcttccaggggtaaaaaaagacgtaatgaaagacatgccccccaaaaaggagctcattttgcgtgttgatctgagcagtctgcagaaagaatattacaaagctatttttacccgtaattatcaagtattgacaaaaaagggaggtgctcaagtaagttctttttaatttttgtttacactttttggatcattaaacctcataggtggggtagaaaccaggtcaactgtaatcgtctagtgaatgtattggtctatttctgtttcagatttcccttaataacattatgatggaattacgaaaagtatgctgccatccttatatgctagagggtgttgagccagttattcacgacgcaaatgaagctttcaagtaatatctcatttcccaaaaatggttatctgtttattactacttattaaagtcgtctgctaacttttgcgttgaacgttttcttatatgtatcaaagacaacttttggagtcttgtggaaagctgcaacttctagataaaatgatggtcaaactgaaagagcaaggacacagagtcctaatatacacacagtttcagcatatgctggacttacttgaagactactgtacccataaggtatttgaacttcttatatgtacagtctgtttcagtagattttcattcttgttgtttttgtagaatatcattttgacactgtagaatcaactctaccattttctagtgttagagtacttaggcacaattatggaaatacaagcatgtgctgaaattgagagtatatgagcattctgtgcccaactgaaagagcaaagacacaaagtttccttataaacacagtacaaatcacaagtttagccatcttctatgtacagtagttttccaataggtcgagcatgtgctgaaactgtgtgtacagagttctcataaacacacagtttcagcatatgctggatctacttgaagactactgttcttataaggtactgaacttgttatctgtactgcgtatatacgagatctctgtattcttgctcttttattttgacactttgttctcatatacactcggttcagcacatgctcgacttactgcctaaggatcttgaaaaaggtagagttgattctatgtctaggtgcaattactttcttagaatttttgtcattacttactctgttggcaatataacttctttattccctcaaagattactttttttggtttcttgaaatgccattatcaataccattgcttttgctgacgcatgcacttgagacaacttgtttttatctctttctagcacatttttttttaacatgcagttaaggaaaattctcatatgatttacgctgttcattttcttgtctttgtcagaaatggcagtacgagcgaattgatggaaaggttggcggagctgagcggcaaatacgcatagatcggttcaatgccaaaaattctaacaagttttgttttttgctctccacaagagctggtggcttaggaataaatcttgcaacggctgatacagtaatcatttatgacaggtttgaatttcagcttctcttagtgtcatctgtactcttttcatagttattgtgtcaagctgtaagaggaactatttggcttgatagcataatattttggaagtttaatgttgatttttaagtgaattgggttgtgatgagtgataaaaaggcacttggcttttttccaataacagctatttcttgaacatggatgttctaagacagcaggaagatcaggaaaattattaaccgctatcttgctaataattagattttgtaggcatgcaatatgggtggcgtccatgggatcctgcttggatggcagtttgttttggtttacgcctgttcacattttcatacgtacgattgaaactgttttatctgtttctgtagtgactggaatcctcatgctgatcttcaagcaatggctagagctcatcgacttggccaaacaaataaggttttaaattttatctcttagtgctgtcaacttgcaattttgtgttcttttttgtagtttccctaattttccttatattttcctttaggtgatgatttataggctcataaaccgaggcaccattgaagaaaggatgatgcaattgactaaaaagaaaatggttctagagcatcttgttgttgggaaactcaaaacacaaaacattaatcaggtaaacttttattgcttgaagcctttttacttgattacaaatttctcaacggattggagctggaaggtagaaattccaagaagaacaccttcggttataacttataagtgtgaaattaaaagataaaaactttagagagaaggggtccatatttgttaattgtttgtcactaagtatgtgtttgttttgttttcctgactgcaatttaggaagagttagatgacatcatcaggtatggatcaaaggagctttttgctagtgaagatgatgaagcaggaaagtctggaaaaattcattatgatgatgcggctatagacaagtaatagactccttactcttttcctcttgttttgtttttgattaacaaggatatctgatctttccgattgctcctttcttatgaaagcttttgcagtcaattgcatgggcgtatttcattatttgtctctatcttctgttctgcagattgcttgatcgtgatctcgtggaggcagaggaagtctcagtggatgatgaagaggagaatggattcttaaaggctttcaaggttttcttgcctcttactattcttcctcttctattagttttctctgaatcagtgtttactgatttcaatgctccattggagtctatgcttaattgtattcttatattccatgatattcagactgtggttggctatcgaaatcccttctgctgtgcacaatcttgtcaaatcattacgtgctaagtttgtaggatcaatacactttatgccagttcgctttgatgcttatagacagtctttagaaagtgtctattgattgttcgttccggctcaatgtgaaagccaacttaatgaaaattagtgatgatgacttaagttagaaatttatgcttgtggtgatgttgattgagccaatttattgatttggttatatttcttttgaaccctgatcatattgaatgcgttatatgagtggtctttagacttagctggaacataaggctgtgtcctgcattgctgcttgtcacctcttaatattcgaactccctaaaacattgtttgtctttgtgtgcatatagaactgttctgaagcaaatagggtgtctggtactgtttagtgtcattaactctgaaaatgatttcccttgtaagattctgtgatcttcctgtattgtaggtggctaattttgaatatatagatgaaaatgaggcagcagcattagaggcacagagagtcgctgctgaaagcaaatcttcagcaggcaattctgatagagcaagttattgggaagagttgttaaaagataaatttgagctgcaccaggctgaggagcttaatgctcttggaaaaaggaagagaagtcgcaagcaggtttggtctcttcttgatcccccttatccaattgtggcatcatattgataactggatttttcaccatttatgttctttctgattctgtcctgtttcatatatttattcatgttgtctaacttttccttttgaattccttaggtagctaaattcagaaagtaataatttagttgactgtatccttctaaattgagaaagtataatttagttgactgtatccagtataaaactaaacgcccttgtcctcctatcaactggtttgacagatcttatgggtttacatgttggatcaagtaattggggttggtagaggctcaattaactatagtcttctgttttcctctgcaagaaatacgttttgtttcactctctaacttgatatagctcaattactgacaatatacattggtttggtctgccatcatcgtttcatgtctttcaataaaggctgttctaattcttctatgggatttttttcatagttggtatccattgaagaagatgatcttgctggtttggaagatgtgagctctgatggagatgaaagttatgaagctgagtcaacagatggtgaagcagcaggacaaggagttcagacgggtcgacggccgtacagaagaaagggtcgcggtattaccacgtttcggatttaatttaatttgtaatggagctgaaaatgactgatattagaagtgtgcgcagtttattagatgagttttttttctatagataatttggaaccaactccgttgatggaaggtgaggggagatctttcagagtactgggtttcaaccagagtcaaagggccatttttgtacagactttgatgaggtatctactttccattaaggcctttagacgccagaagctattctgtctaaattttacagtttcatcccccgatgcatctaaattatcatcagtcttgtggtgctcaatatttacaagtttttccggttggacaaaataattgcaggtatggagctggcaattttgattggaaggagtttgttcctcgcttaaagcagaagacctttgaagaaataaatgagtacgggctcaaccctttaatgctcttctcttctgcttctttacaaaaaacgcatcattataaaaaggctttctggtttattctttaactaattttttaatgactgtttctcagatatggaatactcttcttgaagcacattgctgaagaaatagacgagaattctccaaccttttcaggtgatcgataattgatattttcactgtttgctgcttttccctaaatgagatcattgcttctcctgttaaccggttaaatgtattaatataatggtcgttgtctatagatggtgtgcccaaggaaggacttagaatagaagatgttctagtcagaattgctcttctgatactagttcaggagaaggtgagtctattgactttaattcttcattaagttctctcttttatatctgagtttttttttggtatatgttacttctagtctatagtttagctctgtacataagtttttaatacagtaatgtatgttcaaacctcactaagatttggatcccgggttacttatgtttttttggtgctctggcccgacaggtgaaatttgtagaagatcatccagggaaacctgttttcccctctcgcattcttgaaagattccccggactgagaagtggaaaaatttggaaggaggaacatgacaagataatgatacgtgctgttttaaagtatgaaccctgcaccactgttcttaccgaatggttttattttctcatcattctccattacttgctcacattttcttttccttctctggaaatttgaatctttaggcatgggtacggacggtggcaagctattgttgatgacaaagagttggggatccaagagcttatctgcaaagaattgaatttccctcacataagtttgtctgctgctgaacaagctggtttgcaggggcagaatggtagtgggggctctaatccgggagcacagactaaccagaatcctggaagcgttattactgggaacaataatgcttctgctgatggggctcaagtaaactcgatgttctattatcgggacatgcagagacgacttgttgagtttgtgaaaaagcgagttctgcttttggagaaggcgatgaattatgaatacgcagaggaatattatgtatgttgtaccatctgcagtgttggtacttactcacatgttttgcgctgaattgtttaactttgattgaatctctggttgcagggacttggtggctcatcatctatccctactgaagaaccagaagctgaaccaaagatcgctgacacagtgggagtgagctttattgaggttgatgatgaaatgcttgatggacttcctaagactgatcctatcagtaagttccatcacaagtttctttatttaacgagttgttgattctaatgtgagctctctgaatctcgctgcagcttcagaagaaattatgggggctgctgttgacaacaaccaagcgcgggtcgaaatagctcaacattataaccaggtaagctatgcttttttcctttggtggtaggctaatgtctagaactagtatatcacactaatatctctccggttattcagatgtgcaaacttcttgatgagaacgctcgggaatcagtccaagcatatgtaaacaaccaaccaccgagtaccaaggtgaatgagagcttccgtgcactcaaatctatcaatggtaacattaacacaatcctttcgattacatctgatcaatccaagtcacatgaagacgacaccaagccagacctaaacaatgttgagatgaaggacacggccgaagaaacaaaaccgttaagaggtggcgtcgtcgatctgaatgtggtggagggagaggagaacattgctgaagctagtggaagtgttgatgtaaaaatggaagaagccaaagaagaagagaagccaaagaacatggtcgttgattga

SEQ ID NO: 4 - DNA, 1403 bp, Arabidopsis thaliana
atgaaccttaaggagacggagctttgtcttggcctccccggaggcactgaaaccgttgaaagtccggccaagtcgggtgttgggaacaagagaggcttctccgagaccgttgatctcaaacttaatcttcaatctaacaaacaaggacatgtggatctcaacactaatggagctcccaaggagaagaccttccttaaagacccttctaagcctcctgctaagtaagttctatttacacaattccttaagaagaagaccttccttaaaagggaagactttttttttttttttttgagataaaaagactaatagttgatataaaagttcttaaaatacatatatatgaaagatgtaaggatgcataagtaataacgttattgaatgtgtgtgtgtgttgttatattctatgcagagcacaagtggtgggttggccatcggtgaggaactaccggaaaaatgttatggctaatcagaagagcggcgaagcagaggaggcaatgagtagtggtggaggaaccgtcgcctttgtgaaggtttccatggatggagctccttatcttcggaaggttgacctcaagatgtacaccagctacaaggatctctctgatgccttggccaaaatgttcagctcctttaccatgggtatgcattttcagacatataagtcgaattatcattattatttttgtgtttacttacaattttttctttttaacgatacagttttttccatatacgactaattaatatgataagttttgggattttgattaattaagggagttatggagcacaagggatgatagatttcatgaacgagagtaaagtgatggatctgttgaacagttctgagtatgttccaagctacgaggacaaagatggtgactggatgctcgttggtgatgtcccctggccgtgagtttcctcattcttcttgctttcattattatgaccaaaattattctctaaacaaaaaaaacaatattctctaaagcattattattgatattacttatcaaaaaaatacacaaaatgataatcaatatccatgtgttataaacacgcacagccatcttttggttggcatgggacagaactcagagacagagaagatgtttatatataaatactaactcatcaatatgttacctcatttgtagctggcacatattctttcactttcaatagatttctaaatttagtcaccaacccaaatcccgatttcaggatgtttgtcgagtcatgcaaacgtttgcgcataatgaaaggatccgaagcaattggacttggtaagttttcttttctgttcgtttctataagtggctcttttctgtttttccaataatgctcgtgtttttttttcagctccaagagcaatggagaagttcaagaacagatcatga

SEQ ID NO: 5 - DNA, 21 bp, Artificial Sequence
  synthetic oligonucleotide
catattctgatttaagacata

SEQ ID NO: 6 - DNA, 21 bp, Artificial Sequence
  synthetic oligonucleotide
aatcaatgcatattgtcctct

SEQ ID NO: 7 - DNA, 21 bp, Artificial Sequence
  synthetic oligonucleotide
ttatggctaatcagaagagcg

SEQ ID NO: 8 - DNA, 21 bp, Artificial Sequence
  synthetic oligonucleotide
tattctctaaacaaaaaaaac

SEQ ID NO: 9 - DNA, 21 bp, Artificial Sequence
  synthetic oligonucleotide
aattcgacttctgggtactca

SEQ ID NO: 10 - DNA, 21 bp, Artificial Sequence
  synthetic oligonucleotide
aaattaagtccctcaagctgg

SEQ ID NO: 11 - DNA, 21 bp, Artificial Sequence
  synthetic oligonucleotide
actctgaatttgtagaaagaa

SEQ ID NO: 12 - DNA, 21 bp, Artificial Sequence
  synthetic oligonucleotide
gaagatgattttgttgccata

SEQ ID NO: 13 - DNA, 21 bp, Artificial Sequence
  synthetic oligonucleotide
aagatggggagctggaatatc

SEQ ID NO: 14 - DNA, 21 bp, Artificial Sequence
  synthetic oligonucleotide
ggctcaacaccctctagcata

SEQ ID NO: 15 - DNA, 21 bp, Artificial Sequence
  synthetic oligonucleotide
catccataccagcttgaggga

SEQ ID NO: 16 - DNA, 21 bp, Artificial Sequence
  synthetic oligonucleotide
caagtttgatgtcctcctcac

SEQ ID NO: 17 - DNA, 21 bp, Artificial Sequence
  synthetic oligonucleotide
acatgccccccaaaaaggagc

SEQ ID NO: 18 - DNA, 21 bp, Artificial Sequence
  synthetic oligonucleotide
ccatcaattcgctcgtactgc

SEQ ID NO: 19 - DNA, 21 bp, Artificial Sequence
  synthetic oligonucleotide
atgtgctgaaactgtgtgtac

SEQ ID NO: 20 - DNA, 21 bp, Artificial Sequence
  synthetic oligonucleotide
ccattgcttttgctgacgcat

SEQ ID NO: 21 - DNA, 21 bp, Artificial Sequence
  synthetic oligonucleotide
ttcgatagccaaccacagtct

SEQ ID NO: 22 - DNA, 21 bp, Artificial Sequence
  synthetic oligonucleotide
ggcatgcaatatgggtggcgt

SEQ ID NO: 23 - DNA, 21 bp, Artificial Sequence
  synthetic oligonucleotide
tcaggtatggatcaaaggagc

SEQ ID NO: 24 - DNA, 21 bp, Artificial Sequence
  synthetic oligonucleotide
ctcccctcaccttccatcaac

SEQ ID NO: 25 - DNA, 21 bp, Artificial Sequence
  synthetic oligonucleotide
gtgcacaatcttgtcaaatca

SEQ ID NO: 26 - DNA, 21 bp, Artificial Sequence
  synthetic oligonucleotide
gaggcacagagagtcgctgct

SEQ ID NO: 27 - DNA, 21 bp, Artificial Sequence
  synthetic oligonucleotide
tatacattggtttggtctgcc

SEQ ID NO: 28 - DNA, 21 bp, Artificial Sequence
  synthetic oligonucleotide
gtagggatagatgatgagcca

SEQ ID NO: 29 - DNA, 21 bp, Artificial Sequence
  synthetic oligonucleotide
ccccgatgcatctaaattatc

SEQ ID NO: 30 - DNA, 21 bp, Artificial Sequence
  synthetic oligonucleotide
actagttcaggagaaggtgag

SEQ ID NO: 31 - DNA, 21 bp, Artificial Sequence
  synthetic oligonucleotide
acatgcagagacgacttgttg

SEQ ID NO: 32 - DNA, 21 bp, Artificial Sequence
  synthetic oligonucleotide
cggacttcatcgaacctattc

(19) United States
(12) Patent Application Publication
(10) Pub. No.: US 2004/0244079 A1
(43) Pub. Date: Dec. 2, 2004

(54) Protein that has a function of maintaining a mutation whereby lateral root formation is blocked and a gene encoding the protein

(76) Inventors: Masao Tasaka, Ikoma-shi (JP); Hidehiro Fukaki, Ikoma-shi (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/718,513

(22) Filed: Nov. 24, 2003

(30) Foreign Application Priority Data
May 26, 2003 (JP) 2003-147765

(51) Int. Cl. A01H 5/00; C07H 21/04; C07K 14/00

(52) U.S. Cl. 800/306; 435/410; 536/23.6; 530/350

(57) ABSTRACT

An Arabidopsis thaliana double mutant ssl2 slr having a mutation in at least one base of the SSL2 genomic gene shown in SEQ ID NO: 3, obtained by: treating an Arabidopsis thaliana slr dominant mutant (FERM BP-8385), which has no lateral roots, with a mutagen; preparing plants of the next generation of the mutagen-treated slr dominant mutant; and selecting a plant that basically preserves phenotypes of the slr dominant mutant but has lateral roots from the plants of the next generation.

### CROSS-REFERENCE TO RELATED APPLICATIONS

This application is based upon and claims the benefit of priority from the prior Japanese Patent Application No. 2003-147765, filed May 26, 2003, the entire contents of which are incorporated herein by reference.

### BACKGROUND OF THE INVENTION

1. Field of the Invention

The present invention relates to a factor which has an influence on the phenotype of plants that have no lateral roots (hereinafter also referred to as “lateral rootless phenotype”). The factor is expected to be applicable to artificial control of lateral root formation in plants. More specifically, the present invention relates to a protein having a function of maintaining the mutation whereby lateral root formation is blocked and a gene encoding the protein.

2. Description of the Related Art

The root of dicotyledon plants consists of a primary root which has grown from a radicle of an embryo after germination and lateral roots which have been branched from the primary root. It is known that auxin as a plant hormone is involved in lateral root formation. The SLR (solitary root) gene of Arabidopsis thaliana is also known as a gene encoding a protein which regulates the effect of auxin on lateral root formation. Further, the solitary-root dominant mutant (hereinafter also referred to as “slr dominant mutant”) is known as Arabidopsis thaliana which has a mutation in the SLR gene and forms no lateral roots (Fukaki et al., Plant J. 2002, 29, 153-168). However, there has been no report of a factor which has an influence on the lateral rootless phenotype of the slr dominant mutant and which is expected to be applicable to artificial control of lateral root formation.

### BRIEF SUMMARY OF THE INVENTION

An object of the present invention is to provide a factor which has an influence on the lateral rootless phenotype of a mutant and which is expected to be applicable to artificial control of lateral root formation in plants. More specifically, an object of the present invention is to provide a protein having a function of maintaining the mutation whereby lateral root formation is blocked and a gene encoding the protein.

The present invention may provide the following means for solving the above-mentioned objects.

(1) An Arabidopsis thaliana double mutant ssl2 slr having a mutation in at least one base of the SSL2 genomic gene shown in SEQ ID NO: 3, obtained by: treating (mutagenizing) an Arabidopsis thaliana slr dominant mutant (FERM BP-8385), which has no lateral roots, with a mutagen; preparing plants of the next generation of the mutagen-treated slr dominant mutant; and selecting a plant that basically preserves phenotypes of the slr dominant mutant but has lateral roots from the plants of the next generation.

(2) An Arabidopsis thaliana double mutant ssl2 slr, which has recovered the capability of lateral root formation in an Arabidopsis thaliana slr dominant mutant (FERM BP-8385) that has no lateral roots, due to an additional mutation of at least one base of the SSL2 genomic gene shown in SEQ ID NO: 3 in the slr dominant mutant.

(3) An Arabidopsis thaliana double mutant ssl2 slr, which has recovered the capability of lateral root formation in an Arabidopsis thaliana slr dominant mutant (FERM BP-8385) that has no lateral roots, due to an additional mutation of the SSL2 genomic gene shown in SEQ ID NO: 3 in the slr dominant mutant, wherein the additional mutation is selected from the group consisting of the following (A) to (D):

(A) a mutation in which the 852th base “G” of the SSL2 genomic gene shown in SEQ ID NO: 3 has been substituted with “A”;

(B) a mutation in which the 4734th base “G” of the SSL2 genomic gene shown in SEQ ID NO: 3 has been substituted with “A”;

(C) a mutation in which the 1757th base “G” of the SSL2 genomic gene shown in SEQ ID NO: 3 has been substituted with “A”; and

(D) a mutation in which the 1546th base “G” of the SSL2 genomic gene shown in SEQ ID NO: 3 has been substituted with “A”.

(4) A mutant gene having a mutation in at least one base of the SSL2 gene (cDNA) shown in SEQ ID NO: 1, whose expression enables a phenotype of a mutant that has no lateral roots to be recovered.

(5) A mutant gene having a mutation in at least one base of the SSL2 genomic gene shown in SEQ ID NO: 3, whose expression enables a phenotype of a mutant that has no lateral roots to be recovered.

(6) A mutant gene of the SSL2 gene (cDNA) selected from the group consisting of the following (a) to (c):

(a) a mutant gene in which the 566th base “G” of the SSL2 gene (cDNA) shown in SEQ ID NO: 1 has been substituted with “A”;

(b) a mutant gene in which the 1005th base “G” of the SSL2 gene (cDNA) shown in SEQ ID NO: 1 has been substituted with “A”; and

(c) a mutant gene in which the 901th base “G” of the SSL2 gene (cDNA) shown in SEQ ID NO: 1 has been substituted with “A”.

(7) A mutant gene selected from the group consisting of the following (d) to (g):

(d) a mutant gene in which the 852th base “G” of the SSL2 genomic gene shown in SEQ ID NO: 3 has been substituted with “A”;

(e) a mutant gene in which the 4734th base “G” of the SSL2 genomic gene shown in SEQ ID NO: 3 has been substituted with “A”;

(f) a mutant gene in which the 1757th base “G” of the SSL2 genomic gene shown in SEQ ID NO: 3 has been substituted with “A”; and

(g) a mutant gene in which the 1546th base “G” of the SSL2 genomic gene shown in SEQ ID NO: 3 has been substituted with “A”.

(8) A protein selected from the group consisting of the following (a) and (b):

(a) a protein comprising the amino acid sequence of SEQ ID NO: 2 and having a function of maintaining a mutation whereby lateral root formation is blocked; and

(b) a protein comprising an amino acid sequence of SEQ ID NO: 2, in which one or a few amino acids of the amino acid sequence have been deleted, substituted and/or added and which has a function of maintaining a mutation whereby lateral root formation is blocked.

(9) A gene encoding a protein selected from the group consisting of the following (a) and (b):

(a) a protein comprising the amino acid sequence of SEQ ID NO: 2 and having a function of maintaining a mutation whereby lateral root formation is blocked; and

(b) a protein comprising an amino acid sequence of SEQ ID NO: 2, in which one or a few amino acids of the amino acid sequence have been deleted, substituted and/or added and which has a function of maintaining a mutation whereby lateral root formation is blocked.

(10) A gene selected from the group consisting of the following (c) or (d):

(c) a gene comprising the DNA sequence of SEQ ID NO: 1 and encoding a protein having a function of maintaining a mutation whereby lateral root formation is blocked; and

(d) a gene comprising a DNA sequence of SEQ ID NO: 1, in which one or a few bases of the DNA sequence have been deleted, substituted and/or added and which encodes a protein having a function of maintaining a mutation whereby lateral root formation is blocked.

As described above, the present invention provides a protein having a function of maintaining a mutation whereby lateral root formation is blocked and the SSL2 gene encoding the protein. Further, the inventors of the present invention have found that, when the function of the SSL2 gene of the invention is lost in the slr dominant mutant, the slr dominant mutant loses the lateral rootless phenotype and does form lateral roots. Accordingly, it is assumed that the protein encoded by the SSL2 gene is a novel regulating factor of plant root formation, especially lateral root formation. Thus, it is expected that growth of plant roots can be artificially regulated by modifying the function of the aforementioned protein. Specifically, it is expected to facilitate root formation in an herbaceous or woody plant of various types in which lateral roots or adventitious roots are not formed, by modifying the function of an SSL2-homologous gene in the plant.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

FIG. 1 is a view showing a part of a nucleotide sequence of the SSL2 genomic gene (wild type);

FIG. 2 is a view showing a part of a nucleotide sequence (continued from FIG. 1) of the SSL2 genomic gene (wild type);

FIG. 3 is a view showing a part of a nucleotide sequence (continued from FIG. 2) of the SSL2 genomic gene (wild type);

FIG. 4 is a view showing a part of a nucleotide sequence (continued from FIG. 3) of the SSL2 genomic gene (wild type);

FIG. 5 is a view showing a part of a nucleotide sequence (continued from FIG. 4) of the SSL2 genomic gene (wild type);

FIG. 6 is a view showing a part of a nucleotide sequence (continued from FIG. 5) of the SSL2 genomic gene (wild type);

FIG. 7 is a view showing a part of a nucleotide sequence (continued from FIG. 6) of the SSL2 genomic gene (wild type); and

FIG. 8 is a view showing a nucleotide sequence of a mutant IAA14 gene.

### DETAILED DESCRIPTION OF THE INVENTION

The present invention will be described in detail hereinafter. It should be noted that descriptions below are provided only for illustrating the present invention and do not restrict the present invention.

[Arabidopsis thaliana Double Mutant ssl2 slr]

An Arabidopsis thaliana double mutant ssl2 slr of the present invention (which will be also referred to as “double mutant ssl2 slr” hereinafter) is a double mutant obtained by: treating (mutagenizing) an Arabidopsis thaliana slr dominant mutant, which has no lateral roots, with a mutagen; preparing plants of the next generation of the mutagen-treated slr dominant mutant; and selecting a plant that basically preserves phenotypes of the slr dominant mutant but has lateral roots from the plants of the next generation; wherein the double mutant ssl2 slr has a mutation in at least one base (e.g., one or a few bases) of the SSL2 genomic gene shown in SEQ ID NO: 3.

In another aspect of the present invention, the double mutant ssl2 slr of the present invention is a double mutant which has recovered the capability of lateral root formation in an Arabidopsis thaliana slr dominant mutant that has no lateral roots, due to an additional mutation in at least one base (e.g., one or a few bases) of the SSL2 genomic gene shown in SEQ ID NO: 3 in the slr dominant mutant.

Specifically, the double mutant ssl2 slr of the present invention includes a double mutant which has recovered the capability of lateral root formation in an Arabidopsis thaliana slr dominant mutant that has no lateral roots, by having “a mutant gene of the SSL2 genomic gene” described below in the slr dominant mutant.

In one example, the double mutant ssl2 slr of the present invention is a double mutant which has recovered the capability of lateral root formation in an Arabidopsis thaliana slr dominant mutant that has no lateral roots, by having an additional mutation in the SSL2 genomic gene shown in SEQ ID NO: 3 in the slr dominant mutant, wherein the additional mutation is selected from the group consisting of the following (A) to (D):

(A) a mutation in which the 852th base “G” of the SSL2 genomic gene shown in SEQ ID NO: 3 has been substituted with “A”;

(B) a mutation in which the 4734th base “G” of the SSL2 genomic gene shown in SEQ ID NO: 3 has been substituted with “A”;

(C) a mutation in which the 1757th base “G” of the SSL2 genomic gene shown in SEQ ID NO: 3 has been substituted with “A”; and

(D) a mutation in which the 1546th base “G” of the SSL2 genomic gene shown in SEQ ID NO: 3 has been substituted with “A”.

The Arabidopsis thaliana slr dominant-mutant (which will be also referred to as a “slr dominant mutant” hereinafter), which is used for producing the double mutant ssl2 slr of the present invention, shows a lateral rootless phenotype. The slr dominant mutant also exhibits additional phenotypes in which root hairs are hardly formed and the gravitropism of root and hypocotyl is aberrant. The gene which causes the aforementioned phenotypes including the lateral rootless phenotype in the slr dominant mutant, i.e., SLR mutant gene, will be referred to as “mutant IAA14 gene” hereinafter.

Seeds of the slr dominant mutant have been deposited in the identification name of “solitary-root-1 (Arabidopsis thaliana)” on May 22, 2003, under the International Patent Organism Depositary, National Institute of Advanced Industrial Science and Technology (Tsukuba Central 6, 1-1, Higashi 1-chome, Tsukuba-shi, Ibaraki-ken 305-8566, Japan), pursuant to BUDAPEST TREATY ON THE INTERNATIONAL RECOGNITION OF THE DEPOSIT OF MICROORGANISMS FOR THE PURPOSES OF PATENT PROCEDURE. The accession number “FERM BP-8385” was assigned thereto. The slr dominant mutant can be reproduced by performing self-pollination of the slr dominant mutant, thereby preparing the next generation, and selecting plants that has no lateral roots from the next generation.

In the production of the double mutant ssl2 slr of the present invention, a slr dominant mutant is at first subjected to a mutagen treatment. As the slr dominant mutant to be subjected to the mutagen treatment, seed, plant body, callus and the like may be used. With regard to the mutagen treatment, known techniques may be employed in the present invention. Specific examples of the mutagen include: a chemical mutagen such as an alkylating agent which alkylates bases of DNA; an electromagnetic wave which causes damage to DNA such as X-rays and ultraviolet rays; and a radioactive substance. Alternatively, the mutagen treatment may be effected according to the known Agrobacterium infection method, in which a DNA region sandwiched between a pair of border sequences (25 base pairs) present at both ends of T-DNA region of Ti plasmid contained in Agrobacterium is inserted into a random site of genome DNA of the slr dominant mutant. Preferably, the mutagen treatment is carried out by immersing seeds of the slr dominant mutant for 12 to 16 hours in a solution containing a chemical mutagen (e.g., ethylmethanesulfonic acid) at a concentration of 0.2 to 0.3% by weight. In the case in which seeds are used as the slr dominant mutant, the slr dominant mutants (seeds) are each grown to plants.

Next, the slr dominant mutant (plant body) which has been subjected to the mutagen treatment is made to perform self-pollination and the next generation thereof is produced. Among the thus produced next generation, plants which basically preserve the phenotypes of the slr dominant mutant but form lateral roots (i.e., plants having a mutation caused by the mutagen treatment in a homozygous form) are selected. Here, to “basically preserve the phenotypes of the slr dominant mutant” means maintaining all the characteristics of the slr dominant mutant other than the characteristic of not forming lateral roots. Specifically, “the phenotypes of the slr dominant mutant to be preserved” include a characteristic in which root hairs are hardly formed and a characteristic in which the gravitropism of root and hypocotyl is aberrant.

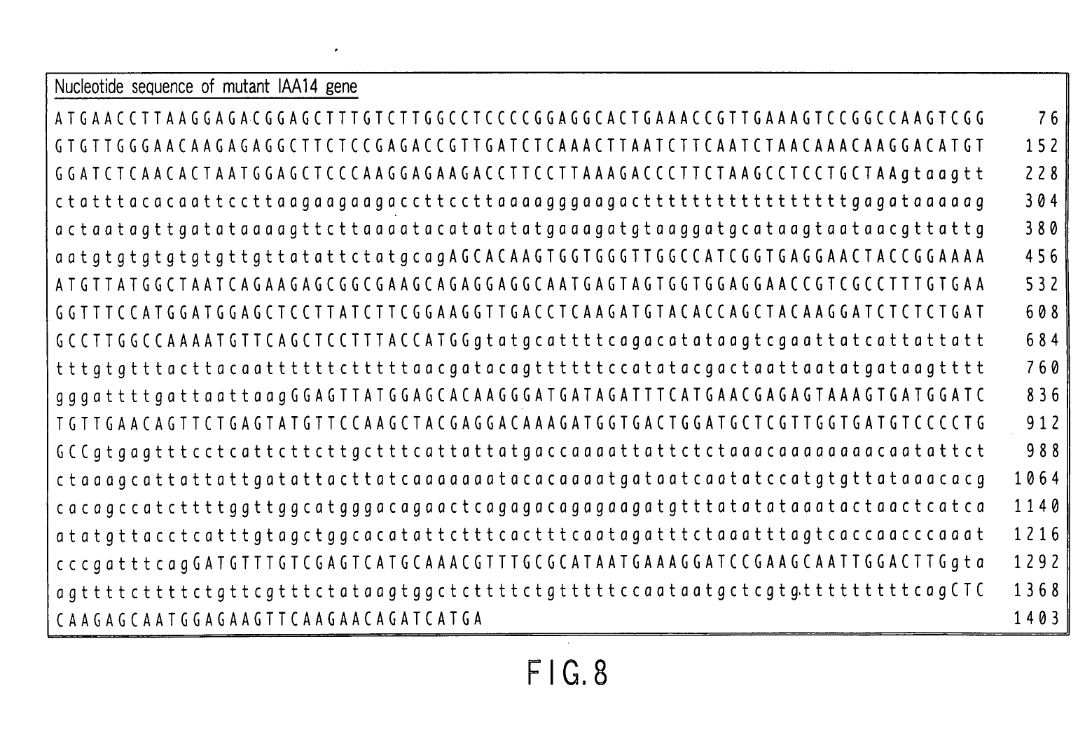

The plants selected at this stage are new mutants which suppress the lateral rootless phenotype of the slr dominant mutant. There is a possibility that these new mutants include two types of mutants: an “intragenic suppressor mutant” in which an additional mutation has occurred inside a region of the gene (mutant IAA14 gene) which causes the mutation of the slr dominant mutant and an “extragenic suppressor mutant” in which an additional mutation has occurred outside the region of the gene (mutant IAA14 gene) which causes the mutation of the slr dominant mutant. Therefore, it is preferable to confirm that the selected plant does not have an additional mutation inside a region of the mutant IAA14 gene. In other words, it is preferable to confirm that the mutation of the mutant IAA14 gene dose not go back to the normal IAA14 gene in the selected plant. With regard to the details of this confirmation, the descriptions of examples described below may be referred to. The information on the nucleotide sequence of the mutant IAA14 gene is available from SEQ ID NO: 4 and FIG. 8. In FIG. 8, the exon portions are indicated by capital letters and the intron portions are indicated by small letters.

The plant selected as described above is the “double mutant ssl2 slr” of the present invention. The double mutant ssl2 slr of the present invention is a double mutant having two mutations: “a slr dominant mutation (originally contained in the slr dominant mutant)” and “a ssl2 recessive mutation (newly caused by the mutagen treatment in the present invention)”. In the present invention, four types of lines (ssl2-1, ssl2-2, ssl2-3 and ssl2-4) were selected as the double mutant ssl2 slr. It has been found that all of the four types of lines have an additional mutation inside the region of the same gene (which will be referred to as “SSL2 genomic gene” hereinafter).

Accordingly, the production of the double mutant ssl2 slr of the present invention is reproducible as described below. That is, plants which form lateral roots are selected from the next generation of the slr dominant mutants which have been subjected to a mutagen treatment; and it is confirmed that the selected plants do not have an additional mutation inside the region of the mutant IAA14 gene and that the selected plants have an additional mutation in the nucleotide sequence of the SSL2 genomic gene. With regard to the technique by which the mutation in the SSL2 genomic gene is confirmed, the descriptions of examples described below may be referred to.

Any of the double mutants ssl2 slr of the present invention exhibit at least some recovery of the phenotypes of the slr dominant mutant. That is, in any of the double mutants ssl2 slr, the lateral rootless phenotype are recovered to form lateral roots, but no recovery is observed in the other phenotypes of the slr dominant mutant (i.e., aberration of root hair formation and aberration of gravitropism). From this fact, it is assumed that the gene (SSL2 genomic gene) which has been mutated in the double mutant ssl2 slr of the present invention genetically interacts with the mutant gene (mutant IAA14 gene) of the slr dominant mutant.

The double mutant ssl2 slr can be reproduced by performing self-pollination of the double mutant ssl2 slr, thereby preparing seeds of the next generation. However, a large number of the seeds of the next generation are not stably obtained, because of the undesirable characteristics of the reproductive organs of the double mutant.

[SSL2 Gene and Protein Encoded by SSL2 Gene]

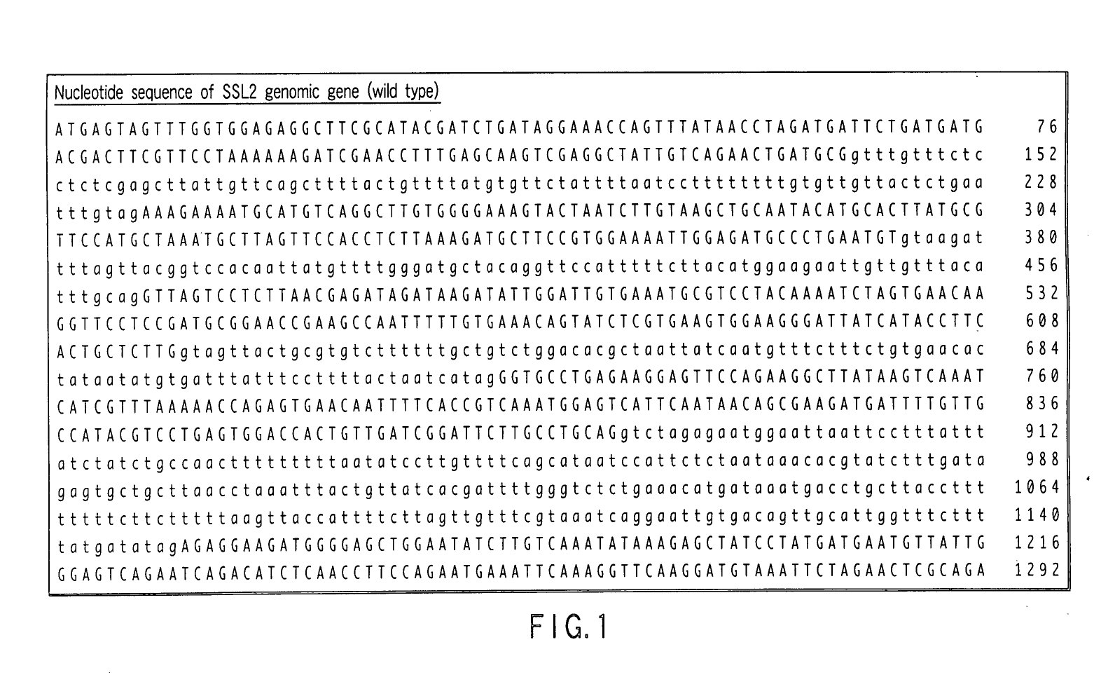

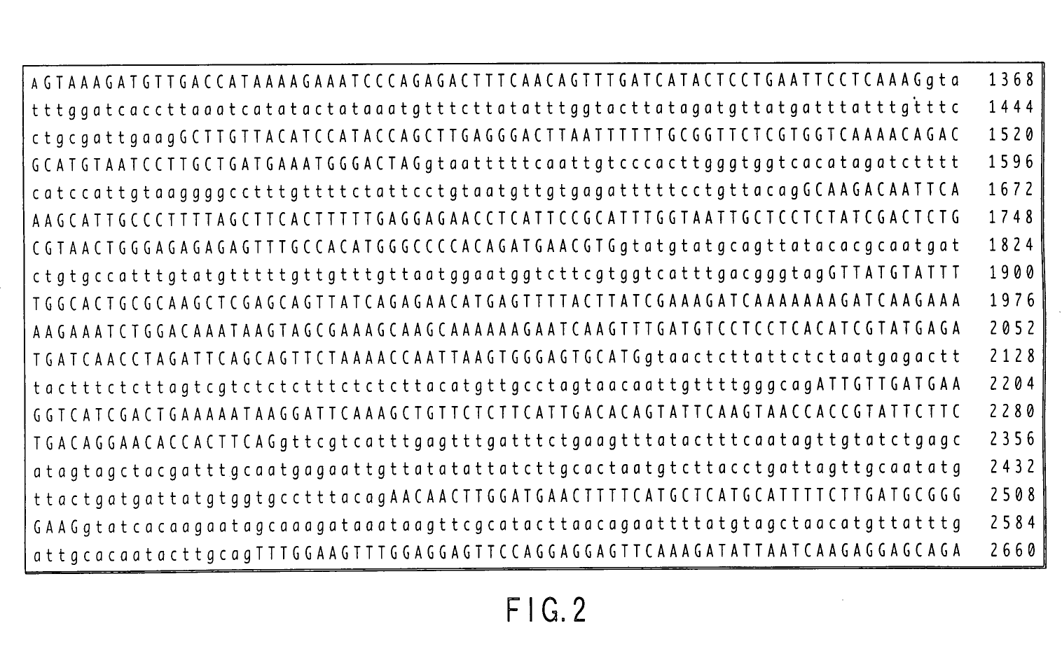

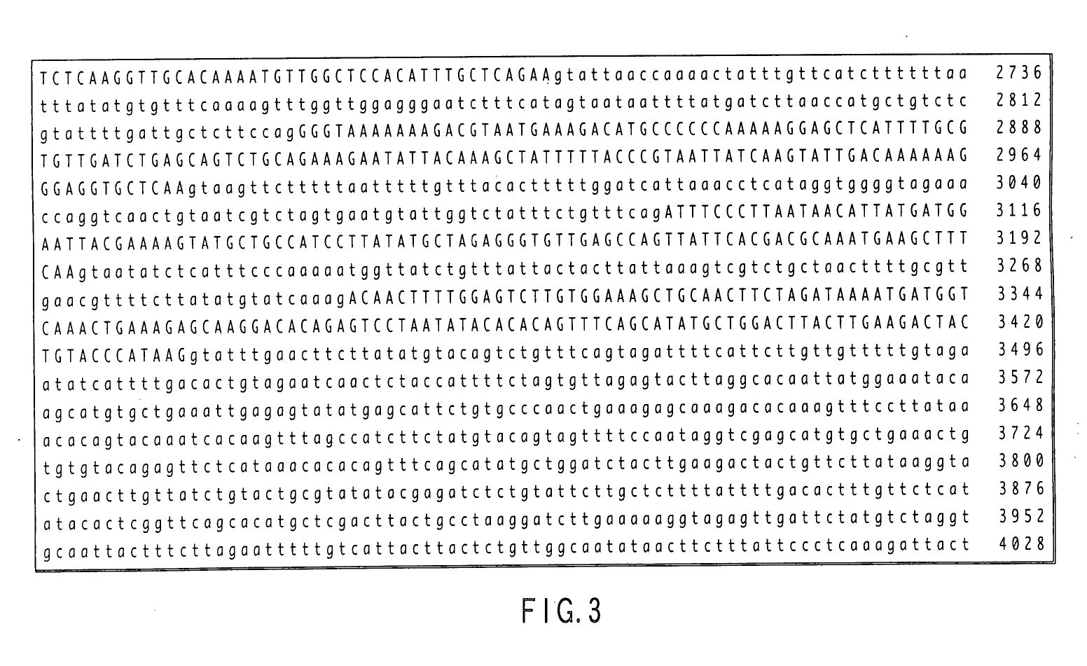

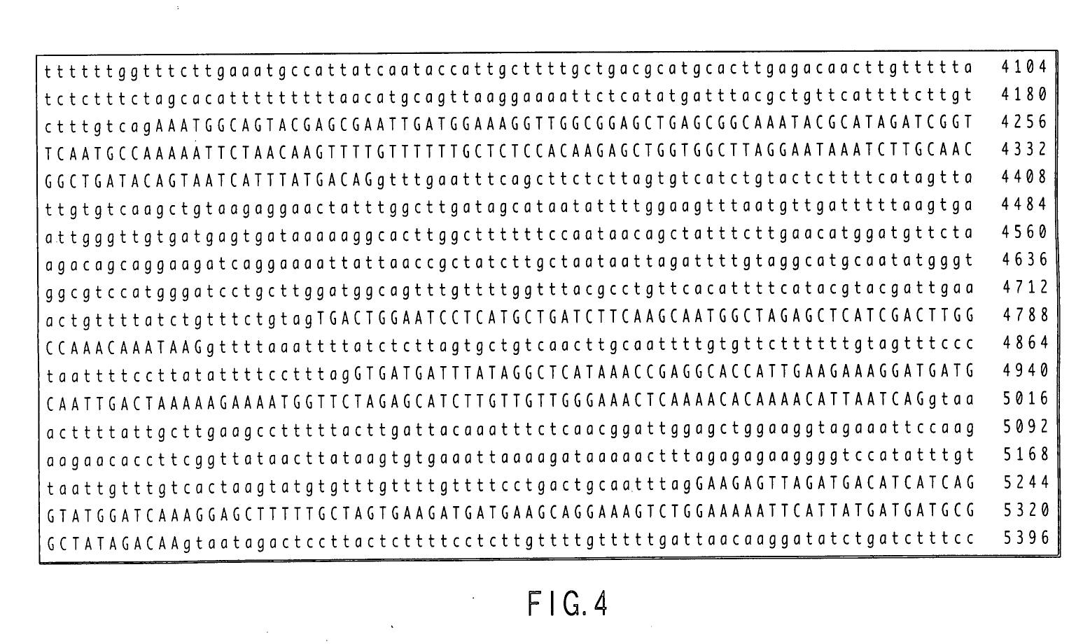

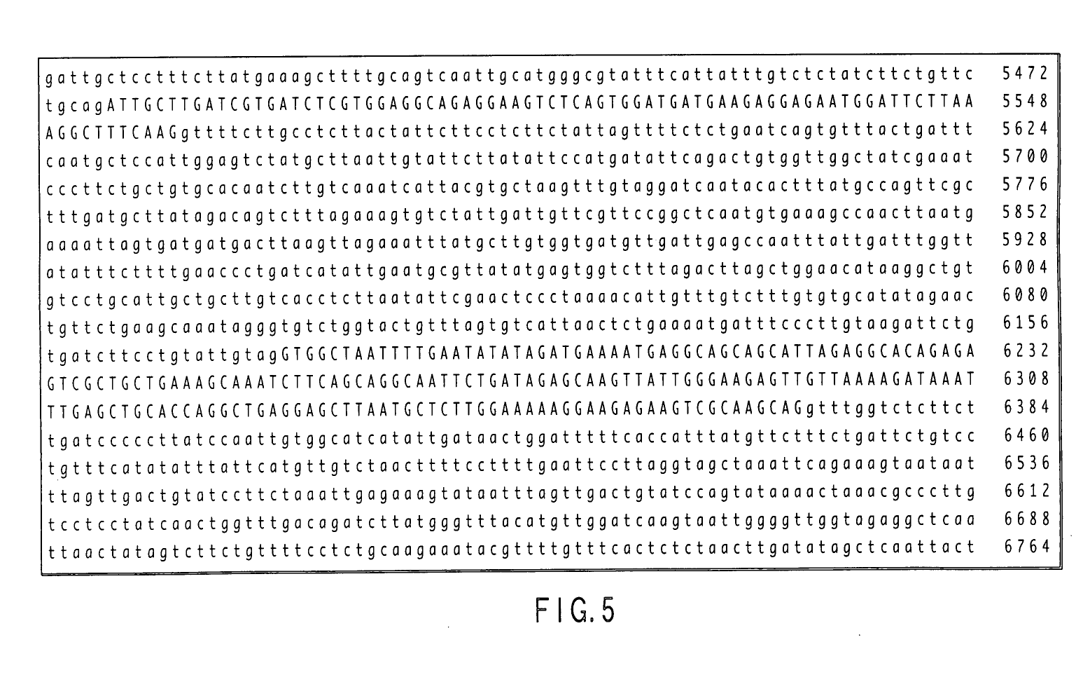

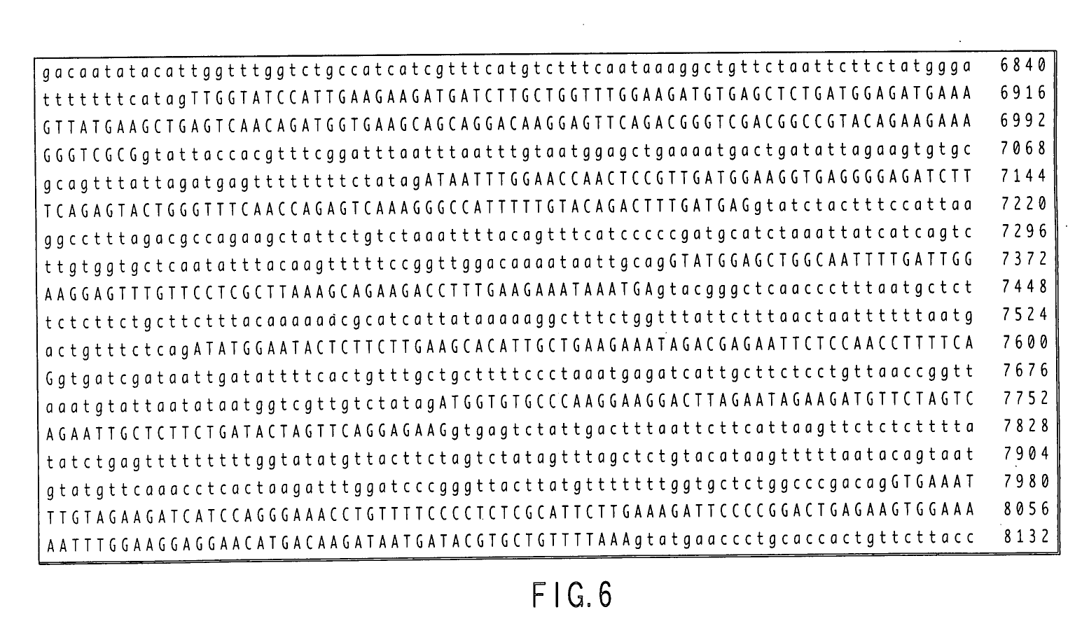

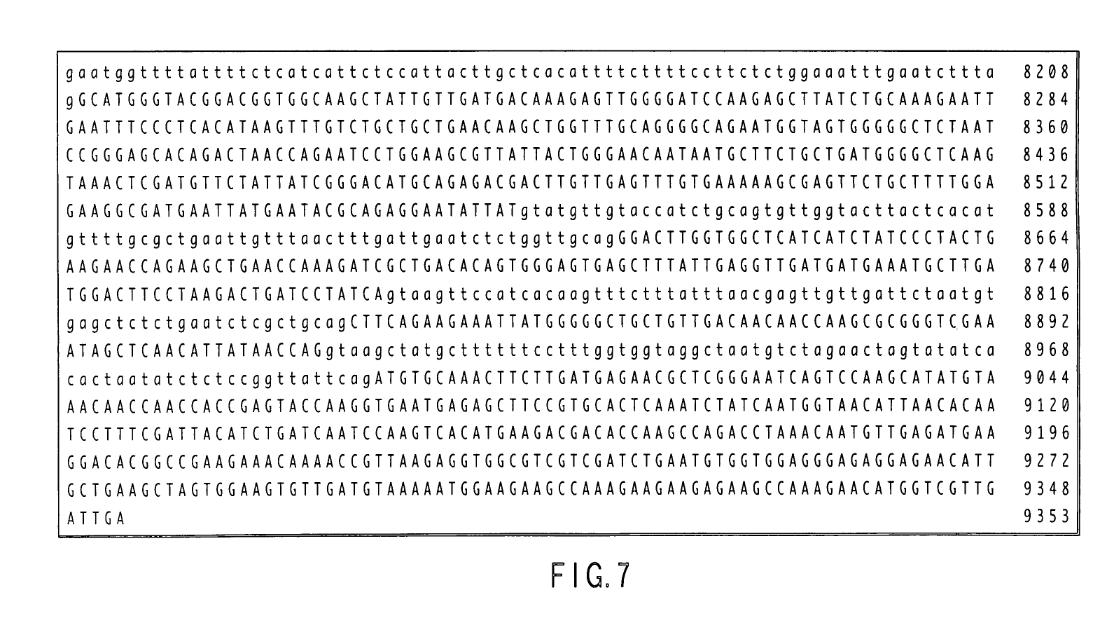

The gene (SSL2 genomic gene) which has been mutated in the double mutant ssl2 slr of the present invention has been identified as At2g25170 gene, according to the mutation map-based cloning for Arabidopsis thaliana. The genetic information on At2g25170 gene is available from the following web page: http://mips.gsf.de/cgi-bin/proj/thal/search_gene? code=At2g25170. It has been confirmed by the present invention that the information on the nucleotide sequence, which was available from the aforementioned web page at the time of filing the present application, is correct but the information from the same source on exon and intron includes errors. Specifically, the inventors of the present invention isolated cDNA of the SSL2 gene which has been mutated in the double mutant ssl2 slr, confirmed the nucleotide sequence thereof, and revealed the correct exon and intron structures of the SSL2 genomic gene (refer to FIGS. 1 to 7).

The nucleotide sequence of the SSL2 gene (cDNA) is shown in SEQ ID NO: 1 and the amino acid sequence of a protein encoded by the SSL2 gene (cDNA) is shown in SEQ. ID NO: 2. The nucleotide sequence of the SSL2 genomic gene is shown SEQ ID NO: 3. Any nucleotide sequence indicates those not having mutation. The nucleotide sequence of the SSL2 genomic gene (SEQ ID NO: 3) is also shown in FIGS. 1 to 7. In FIGS. 1 to 7, the exon portions are indicated by capital letters and the intron portions are indicated by small letters.

As a result of a mutation of the SSL2 genomic gene, the slr dominant mutant becomes to form lateral roots. In other words, the normal SSL2 gene is essential for maintaining mutation whereby lateral root formation is blocked in the slr dominant mutant. Thus, it has been revealed for the first time, by the present invention, that the SSL2 gene encodes a protein having a function of maintaining a mutation whereby lateral root formation is blocked.

Accordingly, the present invention provides a gene comprising the DNA sequence shown in SEQ ID NO: 1 and encoding a protein having a function of maintaining a mutation whereby lateral root formation is blocked. In this gene, one base or a few bases in the DNA sequence shown in SEQ ID NO: 1 may be deleted, substituted and/or added, as long as the gene encodes a protein having a function of maintaining a mutation whereby lateral root formation is blocked.

Further, the present invention provides a gene encoding the following protein: a protein comprising the amino acid sequence shown in SEQ ID NO: 2 and having a function of maintaining a mutation whereby lateral root formation is blocked. Regarding this gene, one amino acid or a few amino acids in the amino acid sequence of the aforementioned protein may be deleted, substituted and/or added, as long as the gene encodes a protein having a function of maintaining a mutation whereby lateral root formation is blocked.

Yet further, the present invention provides a protein comprising the amino acid sequence shown in SEQ ID NO: 2 and having a function of maintaining a mutation whereby lateral root formation is blocked. Regarding this protein, one amino acid or a few amino acids in the amino acid sequence shown in SEQ ID NO: 2 may be deleted, substituted and/or added, as long as the protein has a function of maintaining a mutation whereby lateral root formation is blocked.

In addition, in the present invention, it has been revealed that the SSL2 gene encodes a protein homologous with an animal protein “Chromodomain-helicase-DNA-binding 3 (CHD3)” which is involved in the conversion of chromatin structure of a chromosome. No study has been reported of the relationship between lateral root formation and conversion of chromatin structure. It has been, for the first time in the present invention, suggested that the conversion of chromatin structure is involved in lateral root formation.

[Mutant Gene of SSL2 Gene]

The “mutant gene of the SSL2 gene (cDNA)” of the present invention is a mutant gene having a mutation in at least one base of the SSL2 gene (cDNA) shown in SEQ ID NO: 1, whose expression enables a phenotype of a mutant that has no lateral roots to be recovered.

In the “mutant gene of the SSL2 gene (cDNA)”, “mutation” represents, for example, substitution, deletion, or addition of at least one base, which mutation has an influence on the phenotype of a mutant that has no lateral roots. In other words, the expression of the mutant gene having the above-described mutation enables the phenotype of a mutant that has no lateral roots to be recovered.

Specifically, the “mutant gene of the SSL2 gene (cDNA)” of the present invention includes the following mutant genes. However, it should be noted that the “mutant gene of the SSL2 gene (cDNA)” of the present invention is not limited to these specific examples:

1) a mutant gene in which at least one base (e.g., one base or a few bases) of the SSL2 gene (cDNA) has been substituted with base(s) of other type(s), whereby a codon designating an amino acid has been replaced with a termination codon; and

2) a mutant gene in which at least one base (e.g., one base or a few bases) of the SSL2 gene (cDNA) has been substituted with base(s) of other type(s), whereby a codon designating an amino acid of one type has been replaced with a codon designating an amino acid of another type.

More specifically, the “mutant gene of the SSL2 gene (cDNA)” possessed by the double mutant ssl2 slr selected in the present invention includes the following mutant genes. The mutant genes (a) to (c) are derived from the lines ssl2-1, ssl2-3, ssl2-4 of the double mutant ssl2 slr, respectively:

a) a mutant gene in which the 566th base “G” of the SSL2 gene (cDNA) shown in SEQ ID NO: 1 has been substituted with “A”. As a result of this mutation, tryptophan (TGG) as the 189th amino acid in SEQ ID NO: 2 has been replaced with the termination codon (TAG);

b) a mutant gene in which the 1005th base “G” of the SSL2 gene (cDNA) shown in SEQ ID NO: 1 has been substituted with “A”. As a result of this mutation, tryptophan (TGG) as the 335th amino acid in SEQ ID NO: 2 has been replaced with the termination codon (TAG);

c) a mutant gene in which the 901th base “G” of the SSL2 gene (cDNA) shown in SEQ ID NO: 1 has been substituted with “A”. As a result of this mutation, glycine (GGA) as the 301th amino acid in SEQ ID NO: 2 has been replaced with asparagine (AGA).

Further, the “mutant gene of the SSL2 genomic gene” of the present invention is a mutant gene having mutation in at least one base of the SSL2 genomic gene shown in SEQ ID NO: 3, whose expression enables a phenotype of a mutant that has no lateral roots to be recovered.

Specifically, the “mutant gene of the SSL2 genomic gene” of the present invention includes the following mutant genes. It should be noted that the “mutant gene of the SSL2 genomic gene” of the present invention is not limited to these specific examples:

3) a mutant gene in which at least one base (e.g., one base or a few bases) of the exon portion of the SSL2 genomic gene has been substituted with base(s) of other type(s), whereby a codon designating an amino acid has been replaced with a termination codon;

4) a mutant gene in which at least one base (e.g., one base or a few bases) of the exon portion of the SSL2 genomic gene has been substituted with base(s) of other type(s), whereby a codon designating an amino acid of one type has been replaced with a codon designating an amino acid of another type; and

5) a mutant gene in which at least one base (e.g., one base or a few bases) of a splice site of the SSL2 genomic gene has been substituted with base(s) of other type(s), whereby an intron of the SSL2 genomic gene has not been excised in the normal manner.

In the mutant gene, a “splice site” represents a boundary site between exon and intron, i.e., a site at which excision of an intron and recombination of the two exons adjacent to both ends of the intron are carried out during a splicing reaction, any substitution of a base at which splice site disturbs the splicing reaction. Specifically, a splice site includes the donor splice site located at the 5′ end of an intron and the acceptor splice site located at the 3′ end of an intron. Specific examples of the splice site include the conserved sequence “gt” located at the 5′ end of an intron and the conserved sequence “ag” located at the 3′ end of an intron.

More specifically, the “mutant gene of the SSL2 genomic gene” possessed by the double mutant ssl2 slr selected in the present invention includes the following mutant genes. The mutant genes (d) to (g) are derived from the lines ssl2-1, ssl2-2, ssl2-3, ssl2-4 of the double mutant ssl2 slr, respectively:

d) a mutant gene in which the 852th base “G” of the SSL2 genomic gene shown in SEQ ID NO: 3 has been substituted with “A”. As a result of this mutation at the exon portion of the SSL2 genomic gene, tryptophan (TGG) as the 189th amino acid in SEQ ID NO: 2 has been replaced with the termination codon (TAG);

e) a mutant gene in which the 4734th base “G” of the SSL2 genomic gene shown in SEQ ID NO: 3 has been substituted with “A”. As a result of this mutation at the splice site of the SSL2 genomic gene, the intron of the SSL2 genomic gene has not been excised in the normal manner and thus a normal mRNA is not produced;

f) a mutant gene in which the 1757th base “G” of the SSL2 genomic gene shown in SEQ ID NO: 3 has been substituted with “A”. As a result of this mutation at the exon portion of the SSL2 genomic gene, tryptophan (TGG) as the 335th amino acid in SEQ ID NO: 2 has been replaced with the termination codon (TAG);

g) a mutant gene in which the 1546th base “G” of the SSL2 genomic gene shown in SEQ ID NO: 3 has been substituted with “A”. As a result of this mutation at the exon portion of the SSL2 genomic gene, glycine (GGA) as the 301th amino acid in SEQ ID NO: 2 has been replaced with asparagine (AGA).

### EXAMPLES

Hereinafter, the present invention will be described in detail by examples. It should be noted that the present invention is not limited to the descriptions of these examples.

### Example 1

### Production of a Double Mutant ssl2 slr Having ssl2 Recessive Mutation and slr Dominant Mutation

About 5000 seeds of the slr dominant mutant (FERM BP-8385), which forms no lateral roots, were subjected to a mutagen treatment in which the seeds were immersed in 0.2% ethylmethanesulfonic acid (EMS) solution for 16 hours. The mutagen-treated seeds (M1 seeds) were each grown to plants, and self-pollination of the grown plants was performed, thereby preparing the next generation. Among the next generation (30,000 plants), plants which formed lateral roots although they basically maintained the phenotypes of the slr dominant mutant were selected. After confirming that the aforementioned phenotypes of the selected plants was reliably inherited to the next generation of the selected plants, the selected plants were identified as double mutant and named “double mutant ssl2 slr”. In the present example, double mutants ssl2 slr of four lines (i.e., ssl2-1, ssl2-2, ssl2-3 and ssl2-4) were obtained.

Further, it was confirmed on the basis of the nucleotide sequence that the genome DNA of the double mutant ssl2 slr did not have any additional mutation in a region of the gene (mutant IAA14 gene) causing the slr dominant mutation. That is, it was confirmed that the double mutant ssl2 slr did not correspond to an “intragenic suppressor mutant” in which an additional mutation has occurred inside a region of the mutant IAA14 gene. This confirmation was carried out by amplifying the genomic region including the mutant IAA14 gene, by using the PCR primers shown below. The PCR primer sequences for amplifying the genomic region (1476 base pairs) including the mutant IAA14 gene will be described hereinbelow.

| | | |
|---|---|---|
| IAA14-F1: | 5-CATATTCTGATTTAAGACATA-3 | (SEQ ID NO: 5) |
| IAA14-R1: | 5-AATCAATGCATATTGTCCTCT-3 | (SEQ ID NO: 6) |

The following primers were used, in addition to the above-described two primers, in order to determine the entire nucleotide sequence of the PCR product.

| | | |
|---|---|---|
| IAA14-F2: | 5-TTATGGCTAATCAGAAGAGCG-3 | (SEQ ID NO: 7) |
| IAA14-F3: | 5-TATTCTCTAAACAAAAAAAAC-3 | (SEQ ID NO: 8) |

Further, it was confirmed from the nucleotide sequence that the genome DNA of the double mutant ssl2 slr had a mutation in a region of the SSL2 gene. This confirmation was carried out by amplifying the region of the SSL2 gene, by using primers shown below. Specifically, the nucleotide sequence of the SSL2 gene region was determined by: allotting the SSL2 gene region (9353 base pairs) into 7 sub-regions (A to G); effecting amplification by PCR in each of the sub-regions; determining the entire nucleotide sequence of each PCR product; and comparing the entire nucleotide sequence of each PCR product with the genome DNA sequence of the SSL2 gene of the wild type. If any mutation is found, the plant having the mutation is an ssl2 mutant.

The PCR primer sequences for amplifying each sub-region (A to G) of the SSL2 gene region were as follows.

1) PCR primer sequences for amplifying the sub-region (A)

| | | |
|---|---|---|
| SSL2-F1: | 5-aattcgacttctgggtactca-3 | (SEQ ID NO: 9) |
| SSL2-R1: | 5-AAATTAAGTCCCTCAAGCTGG-3 | (SEQ ID NO: 10) |

The following primers were used, in addition to the above-described two primers, in order to determine the entire nucleotide sequence of the PCR product.

| | | |
|---|---|---|
| SSL2-F2: | 5-actctgaatttgtagAAAGAA-3 | (SEQ ID NO: 11) |
| SSL2-F3: | 5-GAAGATGATTTTGTTGCCATA-3 | (SEQ ID NO: 12) |

2) PCR primer sequences for amplifying the sub-region (B)

| | | |
|---|---|---|
| SSL2-F4: | 5-AAGATGGGGAGCTGGAATATC-3 | (SEQ ID NO: 13) |
| SSL2-R2: | 5-GGCTCAACACCCTCTAGCATA-3 | (SEQ ID NO: 14) |

The following primers were used, in addition to the above-described two primers, in order to determine the entire nucleotide sequence of the PCR product.

| | | |
|---|---|---|
| SSL2-F5: | 5-CATCCATACCAGCTTGAGGGA-3 | (SEQ ID NO: 15) |
| SSL2-F6: | 5-CAAGTTTGATGTCCTCCTCAC-3 | (SEQ ID NO: 16) |

3) PCR primer sequences for amplifying the sub-region (C)

| | | |
|---|---|---|
| SSL2-F7: | 5-ACATGCCCCCCAAAAAGGAGC-3 | (SEQ ID NO: 17) |
| SSL2-R3: | 5-CCATCAATTCGCTCGTACTGC-3 | (SEQ ID NO: 18) |

The following primer was used, in addition to the above-described two primers, in order to determine the entire nucleotide sequence of the PCR product.

| | | |
|---|---|---|
| SSL2-F8: | 5-atgtgctgaaactgtgtgtac-3 | (SEQ ID NO: 19) |

4) PCR primer sequences for amplifying the sub-region (D)

| | | |
|---|---|---|
| SSL2-F9: | 5-ccattgcttttgctgacgcat-3 | (SEQ ID NO: 20) |
| SSL2-R4: | 5-ttcgatagccaaccacagtct-3 | (SEQ ID NO: 21) |

The following primer was used, in addition to the above-described two primers, in order to determine the entire nucleotide sequence of the PCR product.

| | | |
|---|---|---|
| SSL2-F10: | 5-ggcatgcaatatgggtgg | (SEQ ID NO: 22) |
| | cgt-3 | |

5) PCR primer sequences for amplifying the sub-region (E)

| | | |
|---|---|---|
| SSL2-F11: | 5-TCAGGTATGGATCAAAGG | (SEQ ID NO: 23) |
| | AGC-3 | |
| SSL2-R5: | 5-CTCCCCTCACCTTCCATC | (SEQ ID NO: 24) |
| | AAC-3 | |

The following primers were used, in addition to the above-described two primers, in order to determine the entire nucleotide sequence of the PCR product.

| | | |
|---|---|---|
| SSL2-F12: | 5-gtgcacaatcttgtcaaa | (SEQ ID NO: 25) |
| | tca-3 | |
| SSL2-F13: | 5-GAGGCACAGAGAGTCGCT | (SEQ ID NO: 26) |
| | GCT-3 | |

6) PCR primer sequences for amplifying the sub-region (F)

| | | |
|---|---|---|
| SSL2-F14: | 5-tatacattggtttggtct | (SEQ ID NO: 27) |
| | gcc-3 | |
| SSL2-R6: | 5-GTAGGGATAGATGATGAG | (SEQ ID NO: 28) |
| | CCA-3 | |

The following primers were used, in addition to the above-described two primers, in order to determine the entire nucleotide sequence of the PCR product.

| | | |
|---|---|---|
| SSL2-F15: | 5-ccccgatgcatctaaatt | (SEQ ID NO: 29) |
| | atc-3 | |
| SSL2-F16: | 5-ACTAGTTCAGGAGAAGgt | (SEQ ID NO: 30) |
| | gag-3 | |

7) PCR primer sequences for amplifying the sub-region (G)

| | | |
|---|---|---|
| SSL2-F17: | 5-ACATGCAGAGACGACTTG | (SEQ ID NO: 31) |
| | TTG-3 | |
| SSL2-R7: | 5-cggacttcatcgaaccta | (SEQ ID NO: 32) |
| | ttc-3 | |

The above-described two primers were used in order to determine the entire nucleotide sequence of the PCR product.

### Example 2

### Isolation of SSL2 Gene

The double mutant ssl2 slr (ecotype: Columbia) having both ssl2 recessive mutation and slr dominant mutation, prepared in Example 1, was crossed with the wild type (ecotype: Landsberg erecta), whereby F1 generation was obtained. Then, F2 generation as the next generation of F1 generation was prepared by performing self-pollination of the F1 generation. By using the genomic DNA of the F2 generation, detailed mapping of the SSL2 gene locus was carried out on the basis of the genomic information of Arabidopsis thaliana. From the result of the mapping, it was found out that the ssl2 recessive mutation is located in a genomic region including 17 genes from gene At2g25140 to gene At2g25300 on the second chromosome.

Next, in the genomic DNA of the selected four lines (ssl2-1, ssl2-2, ssl2-3 and ssl2-4) of the double mutant ssl2 slr, the nucleotide sequences of the above-described 17 candidate genes were examined. As a result, in all of the four lines of the double mutant ssl2 slr, mutation which presumably causes, the protein encoded by At2g25170 gene, to lose the function thereof was found. On the basis of this discovery, the At2g25170 gene was identified as the SSL2 gene.

### Example 3

### Experiment in which it was Confirmed that the SSL2 Gene is Involved in the Blocking of Lateral Root Formation

The slr dominant mutant (FERM BP-8385) is a gain-of-function mutant of the IAA14 gene encoding an auxin-inducible protein, and lateral root formation thereof is completely blocked under normal growth conditions on an agar medium. However, the double mutant ssl2 slr which has both ssl2 recessive mutation and slr dominant mutation, newly prepared in the present invention, formed lateral roots under the same normal conditions, although the formation of lateral roots was not so vigorous as in the wild type. From this result, it was proved that the normal SSL2 gene is essential for maintaining the lateral rootless phenotype (i.e., the blocking of lateral root formation) observed in the slr dominant mutant.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An Arabidopsis thaliana double mutant ssl2 slr having a mutation in at least one base of the SSL2 genomic gene shown in SEQ ID NO: 3, obtained by: treating an Arabidopsis thaliana slr dominant mutant (FERM BP-8385), which has no lateral roots, with a mutagen; preparing plants of the next generation of the mutagen-treated slr dominant mutant; and selecting a plant that basically preserves phenotypes of the slr dominant mutant but has lateral roots from the plants of the next generation.

2. An Arabidopsis thaliana double mutant ssl2 slr, which has recovered the capability of lateral root formation in an Arabidopsis thaliana slr dominant mutant (FERM BP-8385) that has no lateral roots, due to an additional mutation of at least one base of the SSL2 genomic gene shown in SEQ ID NO: 3 in the slr dominant mutant.

3. An Arabidopsis thaliana double mutant ssl2 slr, which has recovered the capability of lateral root formation in an Arabidopsis thaliana slr dominant mutant (FERM BP-8385) that has no lateral roots, due to an additional mutation of the SSL2 genomic gene shown in SEQ ID NO: 3 in the slr dominant mutant, wherein the additional mutation is selected from the group consisting of the following (A) to (D):
(A) a mutation in which the 852th base “G” of the SSL2 genomic gene shown in SEQ ID NO: 3 has been substituted with “A”;
(B) a mutation in which the 4734th base “G” of the SSL2 genomic gene shown in SEQ ID NO: 3 has been substituted with “A”;
(C) a mutation in which the 1757th base “G” of the SSL2 genomic gene shown in SEQ ID NO: 3 has been substituted with “A”; and
(D) a mutation in which the 1546th base “G” of the SSL2 genomic gene shown in SEQ ID NO: 3 has been substituted with “A”.

4. A mutant gene having a mutation in at least one base of the SSL2 gene (cDNA) shown in SEQ ID NO: 1, whose expression enables a phenotype of a mutant that has no lateral roots to be recovered.

5. A mutant gene having a mutation in at least one base of the SSL2 genomic gene shown in SEQ ID NO: 3, whose expression enables a phenotype of a mutant that has no lateral roots to be recovered.

6. A mutant gene of the SSL2 gene (cDNA) selected from the group consisting of the following (a) to (c):
(a) a mutant gene in which the 566th base “G” of the SSL2 gene (cDNA) shown in SEQ ID NO: 1 has been substituted with “A”;
(b) a mutant gene in which the 1005th base “G” of the SSL2 gene (cDNA) shown in SEQ ID NO: 1 has been substituted with “A”; and
(c) a mutant gene in which the 901th base “G” of the SSL2 gene (cDNA) shown in SEQ ID NO: 1 has been substituted with “A”.

7. A mutant gene selected from the group consisting of the following (d) to (g):
(d) a mutant gene in which the 852th base “G” of the SSL2 genomic gene shown in SEQ ID NO: 3 has been substituted with “A”;
(e) a mutant gene in which the 4734th base “G” of the SSL2 genomic gene shown in SEQ ID NO: 3 has been substituted with “A”;
(f) a mutant gene in which the 1757th base “G” of the SSL2 genomic gene shown in SEQ ID NO: 3 has been substituted with “A”; and
(g) a mutant gene in which the 1546th base “G” of the SSL2 genomic gene shown in SEQ ID NO: 3 has been substituted with “A”.

8. A protein selected from the group consisting of the following (a) and (b):
(a) a protein comprising the amino acid sequence of SEQ ID NO: 2 and having a function of maintaining a mutation whereby lateral root formation is blocked; and
(b) a protein comprising an amino acid sequence of SEQ ID NO: 2, in which one or a few amino acids of the amino acid sequence have been deleted, substituted and/or added and which has a function of maintaining a mutation whereby lateral root formation is blocked.

9. A gene encoding a protein selected from the group consisting of the following (a) and (b):
(a) a protein comprising the amino acid sequence of SEQ ID NO: 2 and having a function of maintaining a mutation whereby lateral root formation is blocked; and
(b) a protein comprising an amino acid sequence of SEQ ID NO: 2, in which one or a few amino acids of the amino acid sequence have been deleted, substituted and/or added and which has a function of maintaining a mutation whereby lateral root formation is blocked.

10. A gene selected from the group consisting of the following (c) or (d):
(c) a gene comprising the DNA sequence of SEQ ID NO: 1 and encoding a protein having a function of maintaining a mutation whereby lateral root formation is blocked; and
(d) a gene comprising a DNA sequence of SEQ ID NO: 1, in which one or a few bases of the DNA sequence have been deleted, substituted and/or added and which encodes a protein having a function of maintaining a mutation whereby lateral root formation is blocked.
